(19) United States
(12) Patent Application Publication
(10) Pub. No.: US 2025/0161942 A1
(43) Pub. Date: May 22, 2025

(54) MICROTRANSFER PATTERNING OF MAGNETIC MATERIALS FOR MICROFLUIDIC APPLICATIONS

(71) Applicant: Trustees of Dartmouth College, Hanover, NH (US)

(72) Inventors: John H. Molinski, Hanover, NH (US); John X.j. Zhang, Hanover, NH (US)

(73) Assignee: Trustees of Dartmouth College, Hanover, NH (US)

(21) Appl. No.: 18/730,697

(22) PCT Filed: Jan. 19, 2023
(86) PCT No.: PCT/US2023/011132

§ 371 Date: Jul. 19, 2024

Related U.S. Application Data
(60) Provisional application No. 63/301,716, filed on Jan. 21, 2022.

(51) Int. Cl. B01L 3/00

(52) U.S. Cl. CPC B01L 3/502761; B01L 2200/0668; B01L 2300/087; B01L 2400/043

(57) ABSTRACT

Embodiments of the present disclosure pertain to microfluidic devices that include at least one channel with a surface and a plurality of magnetic materials positioned on the surface. The plurality of magnetic materials include a metallic component and an adhesive component. The adhesive component is directly positioned on the surface. Further embodiments of the present disclosure pertain to methods of using the microfluidic devices to capture one or more analytes from a sample. Such methods generally include flowing the sample and a plurality of magnetic analyte binding agents through at least one channel of a microfluidic device of the present disclosure. Additional embodiments of the present disclosure pertain to methods of making the microfluidic devices of the present disclosure by forming a plurality of magnetic materials within a cast and transferring the formed magnetic materials from the cast onto a surface to form a channel.

### CROSS-REFERENCE TO RELATED APPLICATIONS

This application claims priority to U.S. Provisional Patent Application No. 63/301,716, filed on Jan. 21, 2022. The entirety of the aforementioned application is incorporated herein by reference.

### BACKGROUND

Current microfluidic devices for analyte detection require complex instrumentation, lack rapid and scalable development, and rely on difficult thin-film deposition or printing-based techniques that only allow for thin films to be fabricated. Various embodiments of the present disclosure seek to address the aforementioned limitations.

### SUMMARY

In some embodiments, the present disclosure pertains to a microfluidic device that includes at least one channel with a surface and a plurality of magnetic materials positioned on the surface. The plurality of magnetic materials include a metallic component and an adhesive component. The adhesive component is directly positioned on the surface. In some embodiments, the plurality of magnetic materials are in the form of a pattern that is operational to mix a plurality of magnetic analyte binding agents with a sample containing analytes. In some embodiments, the microfluidic device is also associated with a magnet that is operational to apply a magnetic field to the plurality of magnetic materials.

In some embodiments, the microfluidic device includes a plurality of channels. In some embodiments, each of the plurality of channels includes at least one inlet for receiving a sample, and at least one outlet for ejecting the sample. In some embodiments, each of the plurality of channels is operational to capture a different analyte from a sample.

Further embodiments of the present disclosure pertain to methods of utilizing the microfluidic devices of the present disclosure to capture one or more analytes from a sample. Such methods generally include flowing the sample and a plurality of magnetic analyte binding agents through at least one channel of a microfluidic device of the present disclosure. The plurality of magnetic analyte binding agents bind to analytes in the sample and become magnetically coupled to the plurality of the magnetic materials of the microfluidic device channel. In some embodiments, the analyte capture methods of the present disclosure include one or more of the following steps: (1) flowing the sample and magnetic analyte binding agents through a channel of a microfluidic device; (2) capturing of the analytes in the sample by magnetic analyte binding agents; (3) coupling of magnetic analyte binding agents to magnetic materials; (4) release of magnetic analyte binding agents from the magnetic materials; and (5) release of captured analytes from magnetic analyte binding agents. In some embodiments, the methods of the present disclosure also include steps of identifying and/or purifying the captured analytes.

Additional embodiments of the present disclosure pertain to methods of making a microfluidic device. In some embodiments, the methods of the present disclosure include at least the steps of (1) forming a plurality of magnetic materials within a cast; and (2) transferring the formed magnetic materials from the cast onto a surface to form a channel.

### DESCRIPTION OF THE DRAWINGS

FIG. 1A depicts a microfluidic device according to an aspect of the present disclosure.

FIG. 1B depicts a method of capturing an analyte from a sample according to an aspect of the present disclosure.

FIG. 1C depicts a method of making a microfluidic device according to an aspect of the present disclosure.

FIG. 2 provides an overall device schematic and working flow of a microfluidic device according to an aspect of the present disclosure.

FIGS. 3A-3F provide a schematic for the fabrication of a microfluidic according to an aspect of the present disclosure.

FIGS. 4A-4C illustrate the characterization of a patterning method for three different pattern geometries of a microfluidic device according various aspects of the present disclosure.

FIGS. 5A-5E show simulations for the magnitude of a magnetic field for different microfluidic device designs.

FIG. 6 shows preliminary capture efficiency studies for proxy fluorescent magnetic particles for different microfluidic device designs.

FIGS. 7A-7F show brightfield microscope images of magnetic materials of different microfluidic devices.

FIGS. 8A-8F show confocal microscope images of magnetic materials of different microfluidic devices.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Magnetic patterning within microfluidic devices has been a topic of prior interest allowing for enhanced capture of magnetically tagged analytes. However, current systems rely on thin-film deposition or printing-based techniques of magnetic materials. Such methods allow only thin films (e.g., <10 μm or less) to be fabricated. Moreover, such methods employ complex fabrication techniques.

In current microfluidic devices which have incorporated magnetic patterning to enhance magnetically tagged analyte capture, complex fabrication processes are usually utilized. Namely, many device configurations utilize photolithography and lift-off to pattern deposited metal structures. In other cases, photolithography and electroplating are used in conjunction, which introduces extra processing steps and more specialized preparation steps.

Typical methods for magnetic patterning require multi-stage lithography-based fabrication protocols that often require fine-tuned protocols and manual intervention. Moreover, due to the typical need for photolithography in the fabrication of each device, device throughput is low, which greatly limits scalability.

Accordingly, a need exists for more effective microfluidic devices and methods of making them. Various embodiments of the present disclosure address the aforementioned need.

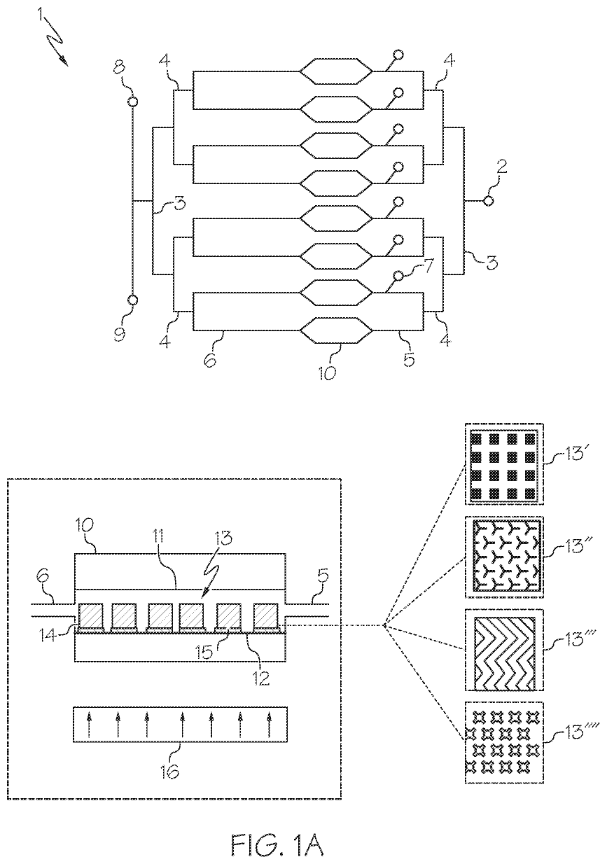

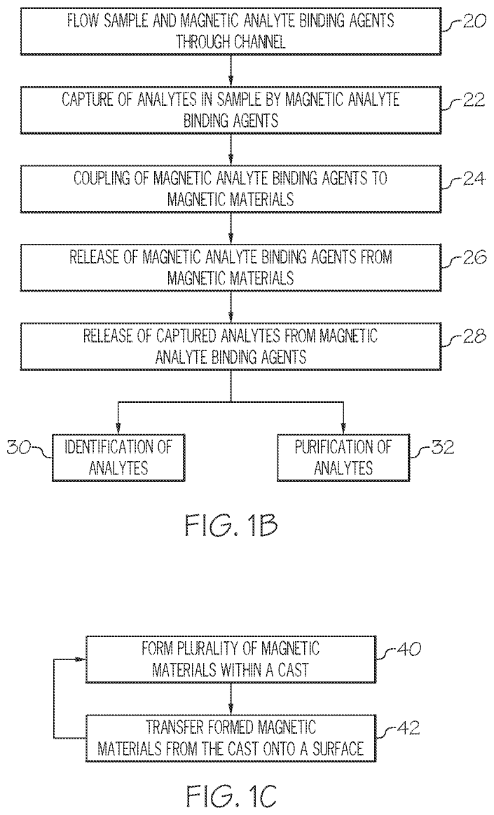

In some embodiments, the present disclosure pertains to microfluidic devices that include a plurality of magnetic materials in a channel. In some embodiments illustrated in FIG. 1A, the microfluidic devices of the present disclosure are represented by microfluidic device 10, which includes at least one channel 11. Channel 11 includes a surface 12 with a plurality of magnetic materials 13 positioned on the surface. Magnetic materials 13 include a metallic component 14 and an adhesive component 15. Adhesive component 15 is directly positioned on surface 12.

As further illustrated in FIG. 1A, magnetic materials 13 may be in the form of various patterns operational to simultaneously mix and capture one or more analytes from a sample. For instance, magnetic materials 13 in the pattern can include shapes of squares 13′, Y-like shapes 13″, herringbones 13″′, X-like shapes 13″″, and combinations thereof.

As also illustrated in FIG. 1A, channel 11 may include at least one inlet 5 for receiving a sample, and at least one outlet 6 for ejecting the sample. Microfluidic device 10 may also be associated with a magnet 16 that is operational to apply a magnetic field to magnetic materials 13.

As further illustrated in FIG. 1A, microfluidic device 10 may be part of a system 1, which includes a plurality of microfluidic devices 10 that are interconnected with flow channels 3 and 4. Each channel 11 within each microfluidic device 10 in system 1 may be operational to capture a different analyte from a sample. In operation, a sample may be introduced into inlet 2 of system 1 such that it flows through each channel 11 of each microfluidic device 10 and flows out of outlets 8 and 9. Sensors 7 may monitor the flow of a sample through each channel 11 of each microfluidic device 10.

Further embodiments of the present disclosure pertain to methods of capturing one or more analytes from a sample. Such methods generally include flowing the sample and a plurality of magnetic analyte binding agents through at least one channel of a microfluidic device of the present disclosure. The plurality of magnetic analyte binding agents bind to analytes in the sample and become magnetically coupled to the plurality of the magnetic materials of the microfluidic device channel. In some embodiments illustrated in FIG. 1B, the analyte capture methods of the present disclosure include one or more of the following steps: flowing the sample and magnetic analyte binding agents through a channel of a microfluidic device (step 20); capturing of the analytes in the sample by magnetic analyte binding agents (step 22); coupling of magnetic analyte binding agents to magnetic materials (step 24); release of magnetic analyte binding agents from the magnetic materials (step 26); and release of captured analytes from magnetic analyte binding agents (step 28). In some embodiments, the methods of the present disclosure also include steps of identifying (step 30) and/or purifying analytes (step 32).

Additional embodiments of the present disclosure pertain to methods of making a microfluidic device. In some embodiments illustrated in FIG. 1C, the methods of the present disclosure include at least the steps of forming a plurality of magnetic materials within a cast (step 40) and transferring the formed magnetic materials from the cast onto a surface to form a channel (step 42).

As set forth in more detail herein, the microfluidic devices of the present disclosure can have numerous embodiments. For instance, the microfluidic devices of the present disclosure can include various surfaces, magnetic materials, channels, heights of the magnetic materials, metallic components, adhesive components, configurations and uses. Furthermore, various methods may be utilized to form the microfluidic devices of the present disclosure. Various methods may also be utilized to capture various analytes from various samples for various purposes by using the microfluidic devices the present disclosure.

### Microfluidic Devices

As set forth in more detail herein, the microfluidic devices of the present disclosure generally include a least one channel. In some embodiments, the at least one channel includes a surface and a plurality of magnetic materials positioned on the surface. In some embodiments, the plurality of magnetic materials include a metallic component and an adhesive component. In some embodiments, the adhesive component is directly positioned on the surface.

As outlined in further detail herein, the microfluidic devices of the present disclosure can include various surfaces, magnetic materials, channels, heights of magnetic materials (e.g., height ratio of the magnetic materials to the channels), metallic components, adhesive components, configurations, and uses.

### Surfaces

The microfluidic devices of the present disclosure can include various types of surfaces. For instance, in some embodiments, the surface is glass. In some embodiments, the surface is a polymer. In some embodiments, the surface is rigid. In some embodiments, the surface is flexible. In some embodiments, the surface is transparent to allow for imaging through devices. In some embodiments, the surface is opaque to block light.

### Magnetic Materials

The microfluidic devices of the present disclosure can include various types and arrangements of magnetic materials. For instance, in some embodiments, the magnetic materials include a plurality of magnetic materials. In some embodiments, the plurality of magnetic materials have a uniform shape. In some embodiments, the plurality of magnetic materials have the same shape. In some embodiments, the plurality of magnetic materials have the same size. In some embodiments, the plurality of magnetic materials have the same structure. In some embodiments, the plurality of magnetic materials have the same shape, size and structure.

In some embodiments, the plurality of magnetic materials are in the form of a pattern. In some embodiments, the pattern is operational to mix a plurality of magnetic analyte binding agents with a sample that includes one or more analytes. In some embodiments, the plurality of magnetic materials in the pattern include one or more shapes. In some embodiments, the shapes include, without limitation, squares, herringbones, Y-like shapes, X-like shapes, and combinations thereof. In some embodiments, the plurality of magnetic materials in the pattern include a herringbone shape.

In some embodiments, the plurality of magnetic materials can be structured either individually or in an array format to modulate the magnetic field and the fluidic behavior local to. In some embodiments, specific geometries of magnetic materials can enhance fluid mixing, bifurcate fluid streamlines, decrease localized fluid velocity, and/or enhance localized magnetic field gradients. In some embodiments, the use of multiple flow channels can enable multifunctionality within a single device, depending on selected use-cases.

In some embodiments, the plurality of magnetic materials form an array. In some embodiments, the arrays are designed to enable additional functionalities, such as mixing within the channel. In some embodiments, the arrays are designed to modulate the magnetic field to specific areas within the channel. In some embodiments, the array design contains elements which are either periodic in two-dimensions or in one dimension. In some embodiments, the arrays act as traps for analytes of interest. In some embodiments, the arrays modulate the fluid flow within the channel and act as filters to certain sized analytes.

In some embodiments, the plurality of magnetic materials have an uneven structure. In some embodiments, the plurality of magnetic materials have different shapes. In some embodiments, the plurality of magnetic materials have different sizes. In some embodiments, the plurality of magnetic materials have different structures.

In some embodiments, the magnetic materials of the present disclosure have a Y shape, a square array shape, a herringbone shape, an X-shape, and combinations thereof. In some embodiments, the magnetic materials of the present disclosure have an X-shape.

### Height of Magnetic Materials

The magnetic materials of the microfluidic devices of the present disclosure can have various heights. For instance, in some embodiments, the magnetic materials are the same height as the channels. In some embodiments, the magnetic materials are the same height as at least one channel. In some embodiments, the height ratio of the magnetic materials to the channel range from 0.2 to 1. In some embodiments, the height ratio of the magnetic materials to the channel range from 0.6 to 1. In some embodiments, the height ratio of the magnetic materials to the channel range from 0.8 to 1. In some embodiments, the height ratio of the magnetic materials to the channel range from 0.9 to 1. In some embodiments, the height ratio of the magnetic materials to the channel is about 0.8.

In some embodiments, the magnetic materials have a height ranging from 5-100% of the channel height. In some embodiments, the magnetic materials have a patterned height in a range of 1 to 100% of the channel height (e.g., 25 μm to 250 μm). In some embodiments, the magnetic materials have a patterned height of less than 1% of the channel height. In some embodiments, the magnetic material height and the channel height have an aspect ratio of 10:1 (height:width).

### Metallic Components

The microfluidic devices of the present disclosure can include various types of metallic components. For instance, in some embodiments, the metallic components are in composite form. In some embodiments, the metallic components are in particle form. In some embodiments, the metallic components include polymers.

In some embodiments, the metallic components include one or more metals. In some embodiments, the one or more metals include, without limitation, metal oxides, metal salts, metal chlorides, metal fluorides, metal bromides, metal iodides, zero valent state metals, multivalent state metals, iron (Fe), cobalt (Co), nickel (Ni), silver (Ag), copper (Cu), gold (Au), platinum (Pt), palladium (Pd), germanium (Ge), and combinations thereof. In some embodiments, the metals are magnetic materials.

### Adhesive Components

The microfluidic devices of the present disclosure can include various types of adhesive components. For instance, in some embodiments, the adhesive components include one or more elastomeric polymers. In some embodiments, the adhesive components include polydimethylsiloxane (PDMS). In some embodiments, the adhesive components adhere to the surface. In some embodiments, the adhesive components are specific to a surface material.

### Channels

The microfluidic devices of the present disclosure can include various types of channels. For instance, in some embodiments, the microfluidic devices include at least one channel. In some embodiments, the microfluidic devices of the present disclosure include a plurality of channels.

In some embodiments, the channels are in an encapsulated form. In some embodiments, the channels include at least on inlet for receiving a sample. In some embodiments, the channels include at least one outlet for ejecting the sample. In some embodiments, the channels include widths ranging from 50 μm to 20,000 μm. In some embodiments, the channels include widths ranging from 1,000 μm to 6,000 μm. In some embodiments, the channels include widths of less than about 15,000 μm. In some embodiments, the channels include widths of less than about 10,000 μm. In some embodiments, the channels include widths of less than about 7,000 μm.

In some embodiments, the channels have heights ranging from 25 μm to 250 μm. In some embodiments, the channels are parallelized to enable multiple analysis simultaneously.

In some embodiments, the microfluidic devices of the present disclosure include a plurality of channels. In some embodiments, each of the plurality of channels are operational to capture a different analyte from a sample. In some embodiments, each of the plurality of channels include at least one inlet for receiving a sample, and at least one outlet for ejecting the sample.

### Magnets and Heating Elements

In some embodiments, the microfluidic devices of the present disclosure are associated with a magnet. In some embodiments, the magnet is operational to apply a magnetic field to the magnetic materials. In some embodiments, the magnetic field can be controlled to preferentially magnetize certain magnetic materials. In some embodiments, the magnetic field is operational to couple magnetic analyte binding agents to magnetic materials.

In some embodiments, the magnet generates an alternating magnetic field. In some embodiments, the alternating magnetic field enables heat generation within the magnetic materials. In some embodiments, the generated heat is operational to lyse captured analytes. In some embodiments, the generated heat is operational to release captured analytes from magnetic analyte binding agents.

In some embodiments, the microfluidic devices of the present disclosure are also associated with a heating element. In some embodiments, the heating element is operational to apply heat to the plurality of magnetic materials.

### Magnetic Analyte Binding Agents

In some embodiments, the microfluidic devices of the present disclosure also include magnetic analyte binding agents. In some embodiments, the magnetic analyte binding agents are operational to magnetically couple to the magnetic materials. In some embodiments, the magnetic analyte binding agents are also operational to bind to an analyte in a sample.

In some embodiments, the magnetic analyte binding agents can include, without limitation, antibodies, aptamers, peptides, peptoids, small molecules, single-stranded nucleic acids, and combinations thereof. In some embodiments, the magnetic analyte binding agents can be modified to express fluorescence or plasmonic behavior. In some embodiments, the size of these magnetic analyte binding agents can be varied from 100 nm to 5 μm in diameter.

### Microfluidic Device Configurations and Uses

The microfluidic devices of the present disclosure can have various configurations. For instance, in some embodiments, the microfluidic devices of the present disclosure may be part of a system that includes a plurality of interconnected microfluidic devices (e.g., system 1 illustrated in FIG. 1A). In some embodiments, each channel within each microfluidic device of a system may be operational to capture a different analyte from a sample.

The microfluidic devices of the present disclosure can be utilized for various applications and uses. For instance, in some embodiments, the microfluidic devices of the present disclosure can be utilized for disease diagnosis. In some embodiments, the microfluidic devices of the present disclosure can be utilized for analyte enrichment. In some embodiments, the microfluidic devices of the present disclosure can be utilized for loading particles. In some embodiments, the microfluidic devices can be used for concentration of analytes within a solution. In some embodiments, the microfluidic devices of the present disclosure can be used for replacing or changing the carrying solution of analytes. In some embodiments, the microfluidic devices of the present disclosure can be used for capture and detection of analytes.

### Methods of Capturing an Analyte

Additional embodiments of the present disclosure pertain to methods of capturing one or more analytes from a sample. Such methods generally include flowing a sample and a plurality of magnetic analyte binding agents through at least one channel of a microfluidic device of the present disclosure. The magnetic analyte binding agents bind to the analytes in the sample and become magnetically coupled to the magnetic materials of the channel. As set forth in more detail herein, the analyte capture methods of the present disclosure can have numerous embodiments.

### Flowing of Samples and Magnetic Analyte Binding Agents Through Channels

Various methods may be utilized to flow samples and magnetic analyte binding agents through channels of the microfluidic devices of the present disclosure. For instance, in some embodiments, the flowing includes flowing a sample and a plurality of magnetic analyte binding agents through a channel of a microfluidic device at the same time. In some embodiments, the flowing includes first flowing a plurality of magnetic analyte binding agents through a channel of a microfluidic device such that the magnetic analyte binding agents become magnetically coupled to the magnetic materials of the channel. Thereafter, a sample is introduced into the channel of the microfluidic device such that one or more analytes in the sample bind to the magnetic analyte binding agents that are coupled to the magnetic materials.

### Samples

The methods of the present disclosure can capture analytes from various samples. For instance, in some embodiments, the sample can include without limitation, a bodily fluid, blood, mucus, fluid from the nasopharynx, a sample from an environment, and combinations thereof. In some embodiments, the sample is obtained from a subject.

### Analytes

The methods of the present disclosure can be utilized to capture various analytes. For instance, in some embodiments, the analytes can include, without limitation, exosomes, cells, circulating tumor cells, particles, metabolites, biomolecules, nucleic acids, circulating nucleic acids, amino acids, peptides, proteins, microbes, viruses, bacteria, yeast, fungi, and combinations thereof. In some embodiments, the analytes can include exosomes.

### Magnetic Analyte Binding Agents

The methods of the present disclosure can utilize various types of magnetic analyte binding agents. For instance, in some embodiments, the magnetic analyte binding agents can include, without limitation, antibodies, aptamers, peptides, peptoids, small molecules, single-stranded nucleic acids, and combinations thereof.

Mixing Samples with Magnetic Analyte Binding Agents

In some embodiments, the methods of the present disclosure can include an additional step of mixing a sample with a magnetic analyte binding agent. In some embodiments, the mixing occurs prior to flowing the sample through a channel of a microfluidic device. In some embodiments, the mixing occurs after flowing a sample through a channel of a microfluidic device.

In some embodiments, the mixing occurs during flowing of a sample through a channel of a microfluidic device. In some embodiments, a pattern of the magnetic materials of a channel mixes the magnetic analyte binding agents with the sample.

### Coupling of Magnetic Analyte Binding Agents to Magnetic Materials

In some embodiments, the methods of the present disclosure also include a step of coupling a plurality of magnetic analyte binding agents to magnetic materials of a channel. In some embodiments, the coupling includes applying a magnetic field to the magnetic materials.

Release of Magnetic Analyte Binding Agents from Magnetic Materials

In some embodiments, the methods of the present disclosure also include a step of releasing magnetic analyte binding agents from magnetic materials of a channel. In some embodiments, the releasing includes removal of a magnetic field from the magnetic materials.

Release of Captured Analytes from Magnetic Analyte Binding Agents

In some embodiments, the methods of the present disclosure also include a step of releasing captured analytes from magnetic analyte binding agents. In some embodiments, the releasing includes removing a magnetic field from the magnetic materials. In some embodiments, the releasing includes washing steps to suspend and remove analytes from magnetic analyte binding agents.

The release of captured analytes from magnetic analyte binding agents can occur at various times. For instance, in some embodiments, the release occurs after the release of magnetic analyte binding agents from magnetic materials. In some embodiments, the release occurs prior to release of magnetic analyte binding agents from magnetic materials. In some embodiments, the release occurs during the release of magnetic analyte binding agents from magnetic materials.

### Identifying Analytes in a Sample

In some embodiments, the methods of the present disclosure also include a step of identifying analytes. In some embodiments, the identifying occurs by methods that can include, without limitation, polymerase chain reactions, utilizing of microarrays, single cell profiling, genomic analysis, proteomic analysis, and combinations thereof.

In some embodiments, the identifying occurs while the analytes are captured by the magnetic analyte binding agents. In some embodiments, the identifying occurs after the analytes are released from the magnetic analyte binding agents.

### Purification of Analytes

In some embodiments, the methods of the present disclosure also include a step of purifying analytes from a sample. For instance, in some embodiments, the purification occurs by releasing the captured analytes from magnetic analyte binding agents. Thereafter, the released analytes may be collected.

### Lysis of Analytes

In some embodiments, the methods of the present disclosure can also include a step of lysing analytes. In some embodiments, the lysing occurs by applying heat to magnetic materials that are coupled to magnetic analyte binding agents and analytes. In some embodiments, the lysing includes applying an alternating current magnetic field to the magnetic materials to cause heating. In some embodiments, the lysing includes flowing a lysis buffer through a channel that includes the magnetic materials.

### Applications

As set forth in further detail herein, the analyte capture methods of the present disclosure can include various applications. For instance, in some embodiments, the methods of the present disclosure can be utilized for disease diagnosis. In some embodiments, the methods of the present disclosure can be utilized for analyte enrichment. In some embodiments, the methods of the present disclosure can be used to concentrate analytes within a solution. In some embodiments, the methods of the present disclosure can be used to enable detection of analytes.

### Methods of Making Microfluidic Devices

Additional embodiments of the present disclosure pertain to methods of making the microfluidic devices of the present disclosure. As detailed herein, such methods generally include: (1) forming a plurality of magnetic materials within a cast; and (2) transferring the formed magnetic materials from the cast onto a surface. In some embodiments, the method can be repeated until the desired number of channels are formed on the surface.

### Forming Methods

The methods of the present disclosure can include various ways of forming magnetic materials within a cast. For instance, in some embodiments, the forming is achieved via heating. In some embodiments, the forming is achieved via curing.

In some embodiments, the forming includes pouring a metallic component into a cast and then pouring an adhesive component onto the metallic component. In some embodiments, the forming includes mixing a magnetic powder and a polymer matrix, packing the mixture into a cast, and then pouring an adhesive component onto the magnetic component.

### Casts

The methods of the present disclosure can utilize various types of casts. For instance, in some embodiments, the cast is in the shape of a mold. In some embodiments, the cast includes anti-sticking components on its surface. In some embodiments, the cast can be fabricated from a flexible polymer material. In some embodiments, the cast can be fabricated from rigid polymer materials. In some embodiments, the cast can be reused repeatedly for patterning.

### Transferring Methods

The methods of the present disclosure can include various methods of transferring the formed magnetic materials from a cast onto a surface. For instance, in some embodiments, the transferring can include flipping. In some embodiments, the transferring can include plasma bonding to increase adhesion to a substrate. In some embodiments, the transferring can include heating to increase adhesion to a substrate. In some embodiments, the transferring includes directly associating an adhesive component with a surface.

### Metallic Components, Adhesive Components, and Magnetic Materials

As set forth in further detail herein, the methods of the present disclosure can utilize various metallic components, adhesive components, and magnetic materials. For instance, in some embodiments, the metallic components can be any metallic component as previously described with respect to the microfluidic devices. In some embodiments, the adhesive components can be any adhesive component as previously described above with respect to the microfluidic devices. In some embodiments, the magnetic materials can be any of the magnetic materials as previously described above with respect to the microfluidic devices.

### Additional Steps

The methods of the present disclosure can include various additional steps. For instance, in some embodiments, the methods of the present disclosure can further include the step of doctor blading a surface to remove excess magnetic materials from the surface. In some embodiments, degassing is used to pack a cast with magnetic materials prior to addition of an adhesion layer. In some embodiments, the magnetic materials are cured after addition of an adhesion layer. In some embodiments, magnetic materials are cured prior to addition of an adhesion layer.

In some embodiments, the methods of the present disclosure also include a step of coupling the formed magnetic materials with at least one inlet for receiving a sample, and at least one outlet for ejecting the sample. In some embodiments, the methods of the present disclosure also include a step of associating the formed magnetic materials with a magnet. In some embodiments, the magnet is operational to apply a magnetic field to the formed magnetic materials.

In some embodiments, the formed magnetic materials include the same shape, size and structure. In some embodiments, the formed magnetic materials are in the form of a pattern. In some embodiments, the plurality of magnetic materials in the pattern include one or more shapes that include, without limitation, squares, herringbones, Y-like shapes, X-like shapes, and combinations thereof. In some embodiments, the formed magnetic materials are arrayed in the form of a channel. In some embodiments, the magnetic material composition can be changed to increase or decrease the magnetic force by adding or removing magnetic materials, respectively. In some embodiments, multiple arrays of magnetic materials of varying magnetic material composition can be fabricated in series with one another, to alter magnetic field and force distributions. In some embodiments, multiple devices can be assembled and connected in series with one another. In some embodiments, each device in a series chain is fabricated with arrays of varying magnetic material composition.

### Advantages and Applications

The microfluidic devices and analyte capture methods of the present disclosure provide numerous advantages. For instance, methods of making the microfluidic devices of the present disclosure utilize convenient fabrication methods and minimal fabrication steps that enable scalable and reproducible fabrication of microfluidic devices.

Additionally, the magnetic materials of the microfluidic devices of the present disclosure can be patterned independent of size, shape, or area. As such, the magnetic materials in the microfluidic devices of the present disclosure can serve dual functions of analyte capture and fluid modulation.

Accordingly, the microfluidic devices and analyte capture methods of the present disclosure can provide numerous applications. For instance, in some embodiments, the microfluidic devices and analyte capture methods of the present disclosure can be utilized in diagnostic applications to capture or detect different biomarkers or analytes in different channels. In some embodiments, the microfluidic devices and analyte capture methods of the present disclosure can be utilized to capture exosomes. In some embodiments, the microfluidic device and analyte capture methods of the present disclosure can be utilized to capture cells and biomolecules, such as circulating tumor cells, and circulating nucleic acids.

Exosomes are a promising biomarker for cancer liquid biopsy because they contain specific molecular components that link it to their parent tumor cells. However, current methods of isolating and detecting exosomes are time consuming, complex, and inefficient. Circulating tumor cells, cells specifically shed from a primary tumor, have long been an intriguing biomarker with applications in minimally invasive cancer diagnostics. Due to their low presence and highly camouflaged nature, there exists challenges in their specific and efficient isolation.

### Additional Embodiments

Reference will now be made to more specific embodiments of the present disclosure and experimental results that provide support for such embodiments. However, Applicants note that the disclosure below is for illustrative purposes only and is not intended to limit the scope of the claimed subject matter in any way.

### Example 1. Microtransfer of Magnetic Materials for Microfluidic Applications

This example illustrates the fabrication and operation of a microfluidic device in accordance with various embodiments of the present disclosure. The microfluidic device presented advantages over current technologies by presenting low-cost and replicable methods for creating magnetically patterned microchips, which enable enhanced immunomagnetic sorting capabilities.

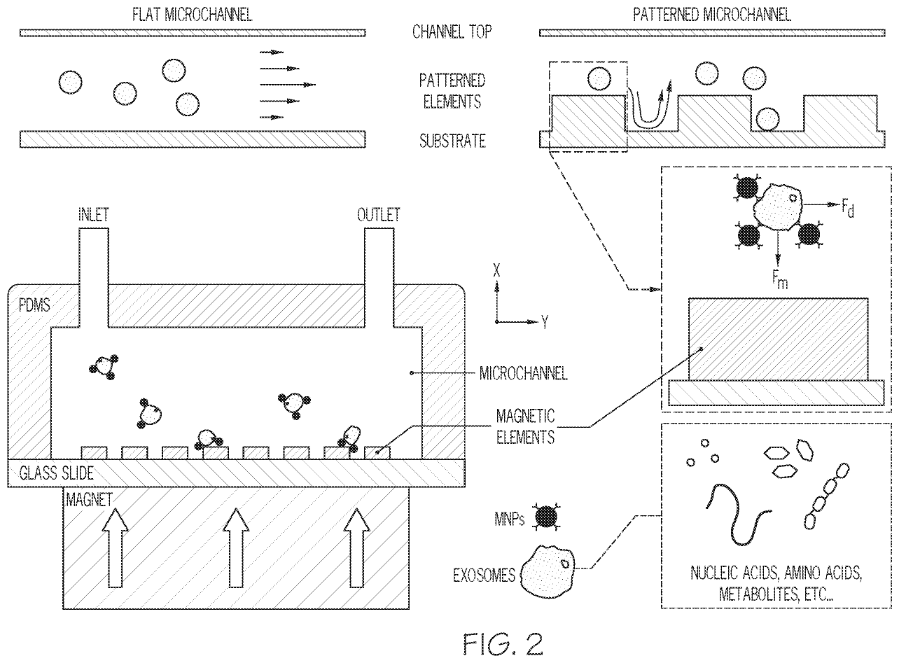

FIG. 2 provides an overall device schematic and working flow of a microfluidic device. This microfluidic device consists of a microfluidic channel encapsulating patterned magnetic elements upon a glass slide or a polymeric substrate. The patterned magnetic elements are fabricated using a modified micro-transfer technique that allows for patterning of magnetic composite materials independent of size, shape or area.

External to the microchip is a permanent magnet which magnetizes the patterned magnetic elements within the microchannel. To capture analytes, analytes are tagged with magnetic particles. Upon flowing through the microchannel, the magnetized magnetic elements attract the nearby particles and allow for trapping and capture. In parallel, by controlling the design and geometry of the pattern, secondary flow modulation such as mixing can occur, thereby increasing probability that tagged analytes interact with capture areas.

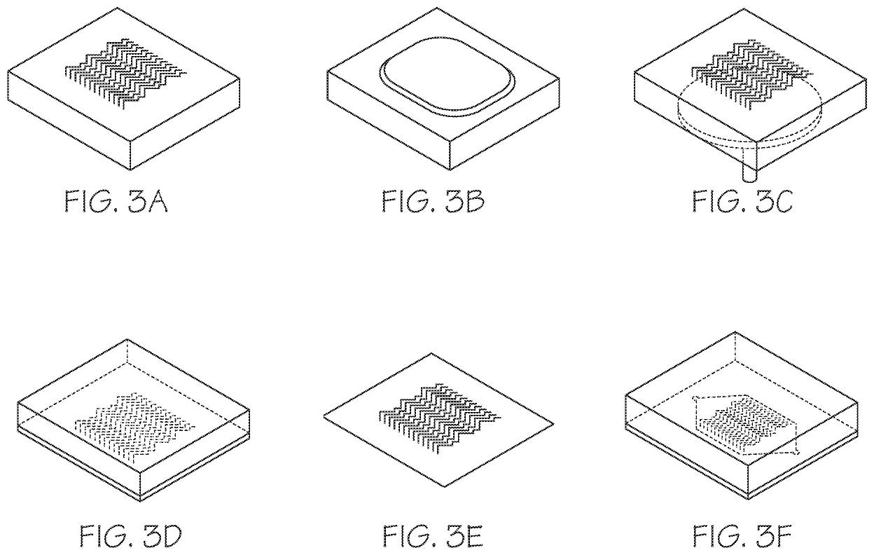

A schematic for the fabrication of the microfluidic device is shown in FIGS. 3A-F. First, a polymeric master mold is fabricated from a silicon master, which defines the pattern for the magnetic patterning (FIG. 3A). Thereafter, a chemical pre-treatment is utilized to create an anti-sticking layer upon the polymer master before the magnetic composite is used to fill the mold, which is then desiccated to remove air, and blade coated such that only the embossed pattern is filled (FIG. 3B). Next, a thin polymeric backing layer is spin coated to add mechanical stability and bind to the uncured magnetic composite (FIG. 3C). After curing both the composite and backing layer in the oven, the polymeric backing layer is plasma treated and adhered to a glass slide or polymer substrate to release the magnetic elements from the polymeric master (FIGS. 3D-3E). Finally, a pre-fabricated channel is bound to encapsulate the magnetic elements (FIG. 3F).

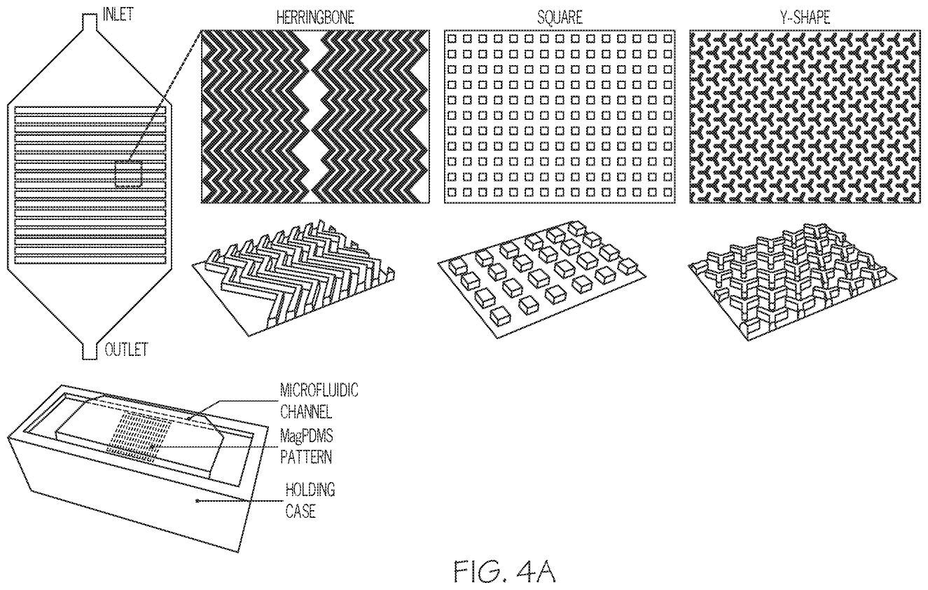

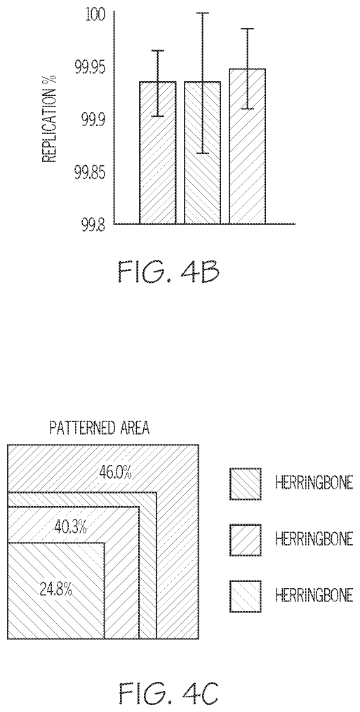

FIG. 4A illustrates the characterization of the patterning method for three different pattern geometries. The illustrations highlight the flexibility and repeatability of the developed approach. The results in FIGS. 4B-4C establish a high replicability rate (FIG. 4B) and a high pattern area (FIG. 4C) for each design, whilst maintaining microscale resolution.

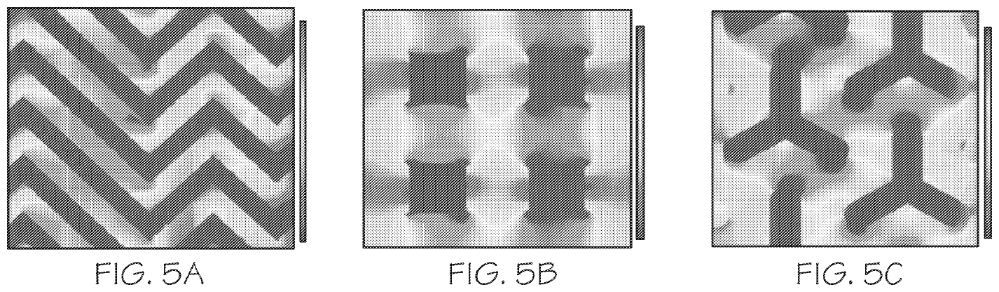

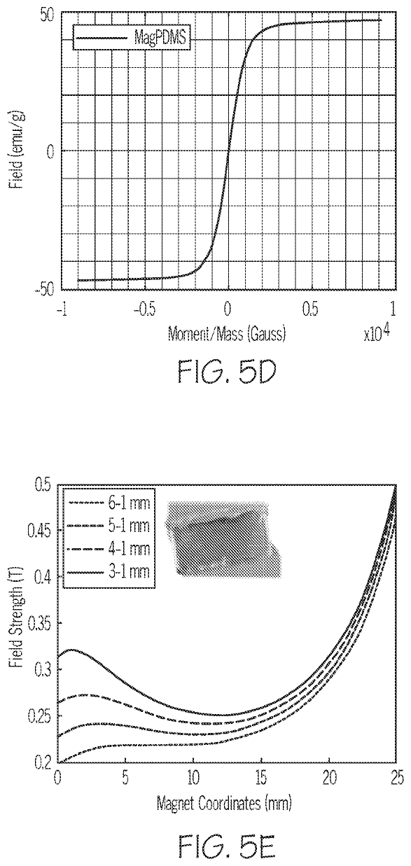

FIGS. 5A-5E show simulations for the magnitude of the magnetic field for the herringbone (FIG. 5A), square (FIG. 5B), and Y-shape (FIG. 5C) designs, which were tested along with the magnetic characterization of the magnetic composite (FIG. 5D). FIG. 5E shows the final image of the distribution of the magnetic field within the channel provided by the bias field.

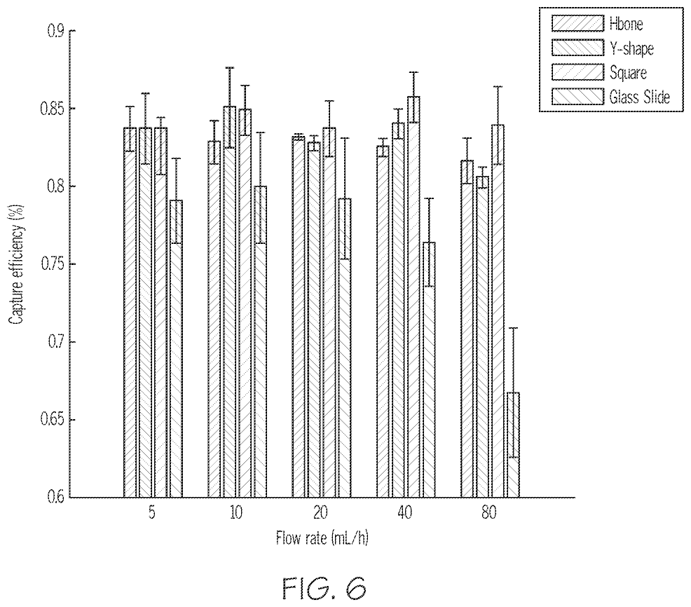

FIG. 6 shows preliminary capture efficiency studies for proxy fluorescent magnetic particles for each of the three designs at different flow rates of 5-80 mL/h. The studies were compared to bare glass slides within a deionized water solution.

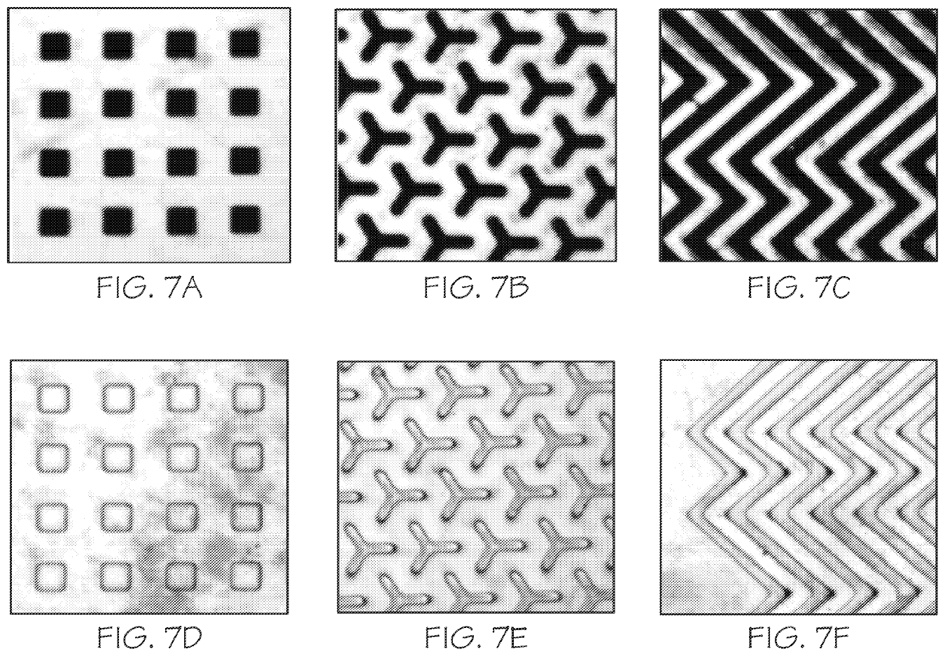

FIGS. 7A-7C show brightfield microscope images for each of the three designs in magnetic composite devices following capture of proxy fluorescent particles. The images were compared to those patterned without magnetic materials (FIGS. 7D-7F).

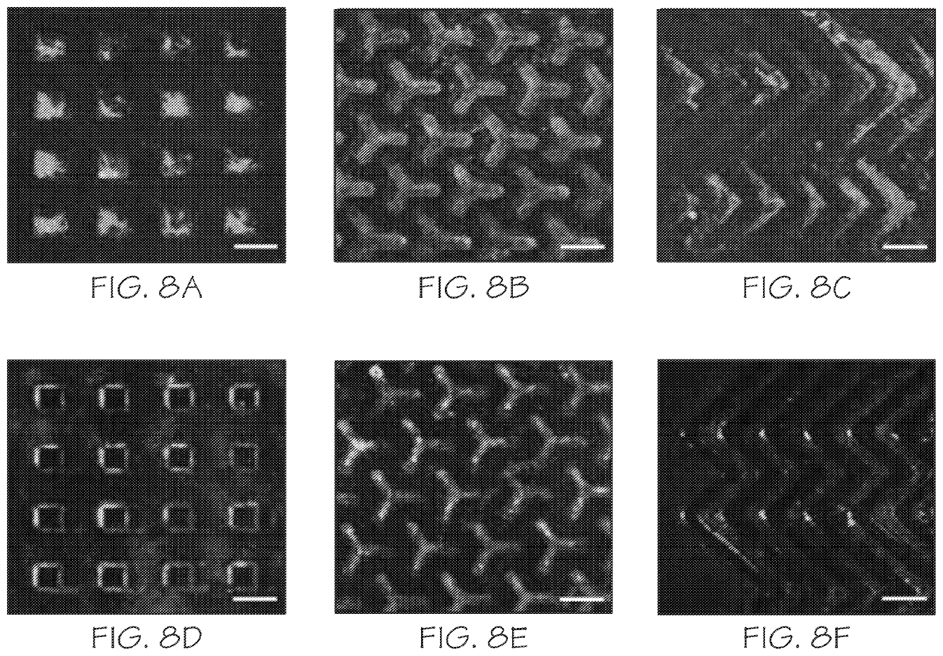

FIGS. 8A-8F show confocal microscope images of each of the three designs in magnetic composite devices, which highlight the preferential capture of the proxy magnetic particles upon the magnetic patterned elements (FIGS. 8A-8C) when compared to those without magnetic patterning (FIGS. 8D-8F).

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A microfluidic device comprising:
at least one channel, wherein the at least one channel comprises:
a surface, and
a plurality of magnetic materials positioned on the surface,
wherein the plurality of magnetic materials comprise a metallic component and an adhesive component, and
wherein the adhesive component is directly positioned on the surface.

2. The microfluidic device of claim 1, wherein the plurality of magnetic materials comprise the same shape, size and structure.

3. The microfluidic device of claim 1, wherein the plurality of magnetic materials are in the form of a pattern, wherein the pattern is operational to mix a plurality of magnetic analyte binding agents with a sample comprising one or more analytes.

4. The microfluidic device of claim 3, wherein the plurality of magnetic materials in the pattern comprise one or more shapes selected from the group consisting of squares, herringbones, Y-like shapes, X-like shapes, and combinations thereof.

5. The microfluidic device of claim 1, wherein the at least one channel is in encapsulated form, and wherein the at least one channel comprises at least one inlet for receiving a sample, and at least one outlet for ejecting the sample.

6. (canceled)

7. The microfluidic device of claim 1, wherein the height ratio of the magnetic materials to the channel ranges from 0.5 to 1.

8. The microfluidic device of claim 1, wherein the metallic component comprises one or more metals selected from the group consisting of metal oxides, metal salts, metal chlorides, metal fluorides, metal bromides, metal iodides, zero valent state metals, multivalent state metals, iron (Fe), cobalt (Co), nickel (Ni), silver (Ag), copper (Cu), gold (Au), platinum (Pt), palladium (Pd), germanium (Ge), magnetic metals, and combinations thereof.

9. The microfluidic device of claim 1, wherein the adhesive component is adhered to the surface.

10. The microfluidic device of claim 1, wherein the adhesive component comprises one or more elastomeric polymers.

11. The microfluidic device of claim 1, wherein the microfluidic device is associated with a magnet, wherein the magnet is operational to apply a magnetic field to the plurality of magnetic materials.

12. The microfluidic device of claim 1, wherein the microfluidic device is associated with a heating element, wherein the heating element is operational to apply heat to the plurality of magnetic materials.

13. The microfluidic device of claim 1, wherein the microfluidic device comprises a plurality of channels, wherein each of the plurality of channels comprises at least one inlet for receiving a sample, and at least one outlet for ejecting the sample, and wherein each of the plurality of channels is operational to capture a different analyte from a sample.

14. The microfluidic device of claim 1, wherein the microfluidic device further comprises magnetic analyte binding agents, wherein the magnetic analyte binding agents are operational to magnetically couple to the plurality of magnetic materials and bind to an analyte in a sample.

15. The microfluidic device of claim 14, wherein the magnetic analyte binding agents are selected from the group consisting of antibodies, aptamers, peptides, peptoids, small molecules, single-stranded nucleic acids, and combinations thereof.

16. A method of capturing one or more analytes from a sample, said method comprising:
flowing the sample and a plurality of magnetic analyte binding agents through at least one channel of a microfluidic device, wherein the microfluidic device comprises at least one channel, wherein the at least one channel comprises:
a surface, and
a plurality of magnetic materials positioned on the surface,
wherein the plurality of magnetic materials comprise a metallic component and an adhesive component, and
wherein the adhesive component is directly positioned on the surface;
wherein the plurality of magnetic analyte binding agents bind to the one or more analytes and become magnetically coupled to the plurality of the magnetic materials of the at least one channel.

17. The method of claim 16, wherein the sample is selected from the group consisting of a bodily fluid, blood, mucus, fluid from the nasopharynx, a sample from an environment, and combinations thereof.

18. The method of claim 16, wherein the one or more analytes is selected from the group consisting of exosomes, cells, circulating tumor cells, particles, metabolites, biomolecules, nucleic acids, circulating nucleic acids, amino acids, peptides, proteins, microbes, viruses, bacteria, yeast, fungi, and combinations thereof.

19. The method of claim 16, wherein the one or more analytes comprises exosomes.

20. The method of claim 16, wherein the plurality of magnetic analyte binding agents are selected from the group consisting of antibodies, aptamers, peptides, peptoids, small molecules, single-stranded nucleic acids, and combinations thereof.

21. The method of claim 16, further comprising a step of mixing the sample with the plurality of magnetic analyte binding agents, wherein the mixing occurs prior to or during the flowing of the sample through the at least one channel of the microfluidic device.

22. (canceled)

23. (canceled)

24. The method of claim 23, wherein a pattern of the plurality of magnetic materials of the at least one channel mixes the plurality of magnetic analyte binding agents with the sample.

25. The method of claim 16, further comprising:
a step of coupling the plurality of magnetic analyte binding agents to the plurality of the magnetic materials, wherein the coupling comprises applying a magnetic field to the plurality of the magnetic materials; and
a step of releasing the plurality of magnetic analyte binding agents from the plurality of the magnetic materials, wherein the releasing comprises removal of a magnetic field from the plurality of the magnetic materials.

26. (canceled)

27. (canceled)

28. (canceled)

29. The method of claim 16, further comprising a step of releasing the one or more analytes from the plurality of magnetic analyte binding agents.

30. The method of claim 29, wherein the releasing comprises a washing step to suspend and remove the one or more analytes from the plurality of magnetic analyte binding agents.

31. The method of claim 16, further comprising a step of identifying the one or more analytes.

32. The method of claim 31, wherein the identifying occurs by a method selected from the group consisting of polymerase chain reactions, utilizing of microarrays, single cell profiling, genomic analysis, proteomic analysis, and combinations thereof.

33. The method of claim 31, wherein the identifying occurs while the one or more analytes are captured by the plurality of magnetic analyte binding agents.

34. The method of claim 31, wherein the identifying occurs after the one or more analytes are released from the plurality of magnetic analyte binding agents.

35. The method of claim 16, further comprising a step of purifying the one or more analytes.

36. The method of claim 35, wherein the purifying comprises releasing the one or more analytes from the plurality of magnetic analyte binding agents.

37. The method of claim 16, further comprising a step of lysing the one or more analytes.

38. The method of claim 37, wherein the lysing comprises applying heat to the plurality of magnetic materials.

39. The method of claim 16, wherein the microfluidic device comprises a plurality of different channels, wherein each channel is utilized to capture a different analyte from the sample.

40. (canceled)

41. The method of claim 16, wherein the flowing comprises:
first flowing the plurality of magnetic analyte binding agents through the at least one channel of the microfluidic device, wherein the plurality of magnetic analyte binding agents become magnetically coupled to the plurality of the magnetic materials of the at least one channel; and
then flowing the sample through the at least one channel of the microfluidic device, wherein the one or more analytes bind to the plurality of magnetic analyte binding agents.

42-52. (canceled)
